(19) United States
(12) Patent Application Publication
(10) Pub. No.: US 2002/0128203 A1
(43) Pub. Date: Sep. 12, 2002

(54) Methods of identifying inhibitory compounds and uses thereof

(76) Inventors: Laurent Schild, Lausanne (CH); Stephan Kellenberger, Lausanne (CH)

Correspondence Address: Ivor R. Elrifi, MINTZ, LEVIN, COHN, FERRIS,, GLOVSKY AND POPEO, P.C., One Financial Center, Boston, MA, 02111, US

(21) Appl. No.: 09/956,522

(22) Filed: Sep. 19, 2001

Related U.S. Application Data
(60) Provisional application No. 60/234,021, filed on Sep. 20, 2000.

(51) Int. Cl. A61K 38/08; G01N 33/53

(52) U.S. Cl. 514/15; 514/16; 435/7.1

(57) ABSTRACT

The invention relates to compositions comprising a sodium channel inhibitor. Also provided are methods of identifying such inhibitors, as well as kits and pharmaceutical compositions containing the same. The invention also relates to methods of treating disorders associated with irregular or improper epithelial sodium channel (“ENAC”) activity, including hypertension, renal insufficiency, electrolyte imbalances, cystic fibrosis, and Liddle's syndrome.

### RELATED APPLICATIONS

This application claims the benefit of U.S. Ser. No. 60/234,021, filed Sep. 20, 2000, which is incorporated herein by reference.

### FIELD OF THE INVENTION

The invention relates generally to molecular biology, and particularly to compounds that inhibit of the epithelial sodium channel (“ENAC”).

### BACKGROUND OF THE INVENTION

Hypertension, classified as an elevated arterial blood pressure, is an important public health problem in developed countries. Although common, readily detectable, and usually easily treatable, hypertension can be lethal if left untreated. Factors such as heredity and the environment can contribute to the genesis of hypertension. Environmental factors may include salt intake, obesity, and occupation. Abnormal high blood pressure also carries additional risks for cardiovascular diseases such as stroke, kidney failure, and myocardial infarction. In some patients, hypertension may be the result of renal disease or adrenal cortical abnormalities. Such forms of hypertension are known as secondary hypertension.

Current treatments for hypertension include non-drug therapeutic interventions such as relief of stress, diet and exercise, and the control of other risk factors. In addition, common drug therapies include diuretics, antiadrenergic agents, vasodilators, angiotensin-converting enzyme inhibitors, and calcium channel antagonists.

### SUMMARY OF THE INVENTION

The invention is based on the discovery of specific target sequences for ligand binding and channel inhibition in the N-termini of an α, β, or γ polypeptide of an ENAC. Inhibitors which preferentially associate with or bind to such target sequences are useful to modulate, e.g., block, the sodium channel for the purpose of reducing hypertension and alleviating the symptoms of renal insufficiency and other diseases associated with water and electrolyte imbalances.

In one aspect, the invention provides sodium channel inhibitors that bind to a polypeptide having the amino acid sequence HGXXRXV (SEQ ID NO:4), HGXXRXXC (SEQ ID NO:5), or HGXXRXXXS (SEQ ID NO:6), wherein X is any amino acid. In one embodiment, this inhibitor binds to a polypeptide having the amino acid sequence HGAIRLVCSQH (SEQ ID NO:1), HGPKRIICEGP (SEQ ID NO:2), or HGCRRIVVSRG (SEQ ID NO:3). In an alternative embodiment, this polypeptide has an amino acid sequence that is at least 70% identical to HGAIRLVCSQH (SEQ ID NO:1), HGPKRIICEGP (SEQ ID NO:2), or HGCRRIVVSRG (SEQ ID NO:3). In still further embodiments, the polypeptide has an amino acid sequence that is at least 80%, at least 85%, at least 90%, at least 95%, at least 96%, at least 97%, at least 98%, at least 99%, or at least 100% identical to these sequences. Collectively, the polypeptides of SEQ ID NOS:1-6 are referred to herein as “ENAC polypeptides”. For example, the inhibitor is an amiloride or methanethiosulfonate. Preferably, the inhibitor is not amiloride or a methanethiosulfonate or analogues of amiloride and triamterene.

By analogue is meant a compound that is similar or comparable, but not identical, to a reference compound, i.e. a compound similar in function and appearance, but not in structure or origin to the reference compound. For example, the reference compound can be a reference channel inhibitor such as amiloride, trimterene or methanethiosulfonate and an analogue is a substance possessing a chemical structure or chemical properties similar to those of the reference channel inhibitor. As used herein, an analogue is a chemical compound that may be structurally similar to another but differs in composition (as in the replacement of one atom by an atom of a different element or in the presence of a particular functional group). Analogues function to reversibly inhibit an ENAC or ASIC. Non-identical amino acids within the sequence are preferably conservative amino acid substitutions.

In another embodiment, the invention includes a pharmaceutical composition containing a sodium channel inhibitor and a pharmaceutically acceptable carrier. The invention also includes a kit containing, in one or more containers, such a composition comprising an ENAC inhibitor packaged together with instructions for their use.

In another aspect, this invention involves a method of identifying a sodium channel inhibitors by contacting an ENAC polypeptide having the amino acid sequence of any one of SEQ ID NOS:1, 2, 3, 4, 5, or 6 with a candidate compound and determining if the candidate compound binds to the polypeptide. Binding of the compound indicates that the compound inhibits ENAC activity. In various embodiments, the candidate compound may be a polypeptide, a polypeptide fragment, a peptide mimetic, a small organic molecule, a large organic molecule, or any other drug candidates known to those skilled in the art. Also provided are sodium channel inhibitors identified according to these methods.

A further aspect of this invention involves a method for reducing hypertension, renal insufficiency, other pathological conditions associated with water or electrolyte imbalance, or disorders associated with irregular or inappropriate ENAC activity in a mammal by administering a compound that binds to an ENAC polypeptide having the amino acid sequence of any one of SEQ ID NOS:1, 2, 3, 4, 5, or 6. The peptide-binding inhibitors defined by the invention may optionally be administered with amiloride or a methanethiosulfonate. Examples of such disorders include, but are not limited to, hypertension, renal insufficiency, electrolyte imbalances, cystic fibrosis, and Liddle's syndrome.

All technical and scientific terms used herein have the same meanings commonly understood by one of ordinary skill in the art to which this invention belongs. Although any methods and materials similar or equivalent to those described herein can be used in the practice of the present invention, the preferred methods and materials are now described. The citation or identification of any reference within this application shall not be construed as an admission that such reference is available as prior art to the present invention. All publications mentioned herein are incorporated by reference in their entireties.

### BRIEF DESCRIPTION OF THE DRAWINGS

FIG. 1a is a diagram depicting the functional domains in ENAC subunit. The four distinct functional domains depicted include: (1) internal gating domain; (2) external gating domain; (3) amiloride binding site; and (4) regulatory site.

FIG. 1b is a diagram showing membrane topology of ENAC subunit. Activating sites are shown in light shading whereas inhibitory sites are shown in dark shading.

FIG. 2 is a diagram showing an alignment of the intracellular N-terminal parts of human and rat ENAC subunits.

### DETAILED DESCRIPTION OF THE INVENTION

Essential hypertension is disorder influenced by both genetic and environmental factors. The kidneys play an important role in the maintenance of sodium balance, extracellular fluid volume, and long-term control of blood pressure, and sodium transporters in the kidney affect the amount of sodium and water reabsorption in the nephron and thus control extracellular fluid volume and blood pressure. Renal sodium transporters, e.g., the amiloride-sensitive epithelial sodium channels or ENaC, are responsible for the rate-limiting step of sodium reabsorption in the distal nephron and play a role in the development of hypertension. For example, mutations in this channel underlie a rare form of heritable hypertension (Liddle's syndrome), and genetic linkage studies indicate that the beta- and gamma-subunits are linked to systolic blood pressure. The invention provides compositions and molecular targets for the development of sodium channel inhibitors, which are useful for the clinical treatment of hypertension.

Sodium Channels and Hypertension

Specific target sequences and target consensus sequences have been identified that are important in blocking the activity of an epithelial sodium channel (ENAC). This discovery provides a method of identifying substances which block ENAC activity. Also disclosed are methods of treating hypertension, renal insufficiency, any electrolyte imbalance, cystic fibrosis, Liddle's syndrome, as well as any other diseases and/or syndromes that are caused by or result from irregular or inappropriate ENAC activity.

A sodium channel is a cell membrane channel capable of conducting sodium from an extracellular space into the cytoplasm of a cell. Movement of sodium ions is dependent upon the electrical potential of the cell membrane. An open or activated sodium channel is one that conducts sodium ions.

In certain tissues, sodium reabsorption is mediated by an amiloride-sensitive electrogenic sodium transport through the amiloride-sensitive epithelial sodium channel (“ENAC”). The ENAC is highly sodium-selective, and, thus, does not allow the entry or exit of any potassium ions. ENAC is a heteromultimeric protein composed of three homologous subunits. Each of the α, β, and γ subunits vary in length from 650 to 700 amino acids. At the protein level, each subunit shares 35% amino acid identity with the others.

ENAC proteins are expressed in low copy number, and, thus, typically, only a few hundred molecules are expressed per cell. Additionally, ENAC protein tissue distribution is restricted to a few organs including the apical membranes of aldosterone-responsive tissues (i.e., the distal part of the nephron of the kidney, the distal part of the colon, and the ducts of exocrine glands); the epidermis of the skin; in hair follicles; the lungs; and the nephron. The ENAC regulates fluid and electrolyte transport and maintains Na+and fluid homeostasis in the body.

Inhibitory Compounds

A sodium channel inhibitor is a compound which decreases the conductance of sodium ions through a sodium channel. Examples of known sodium channel inhibitors include amiloride and the methanethiosulfonates.

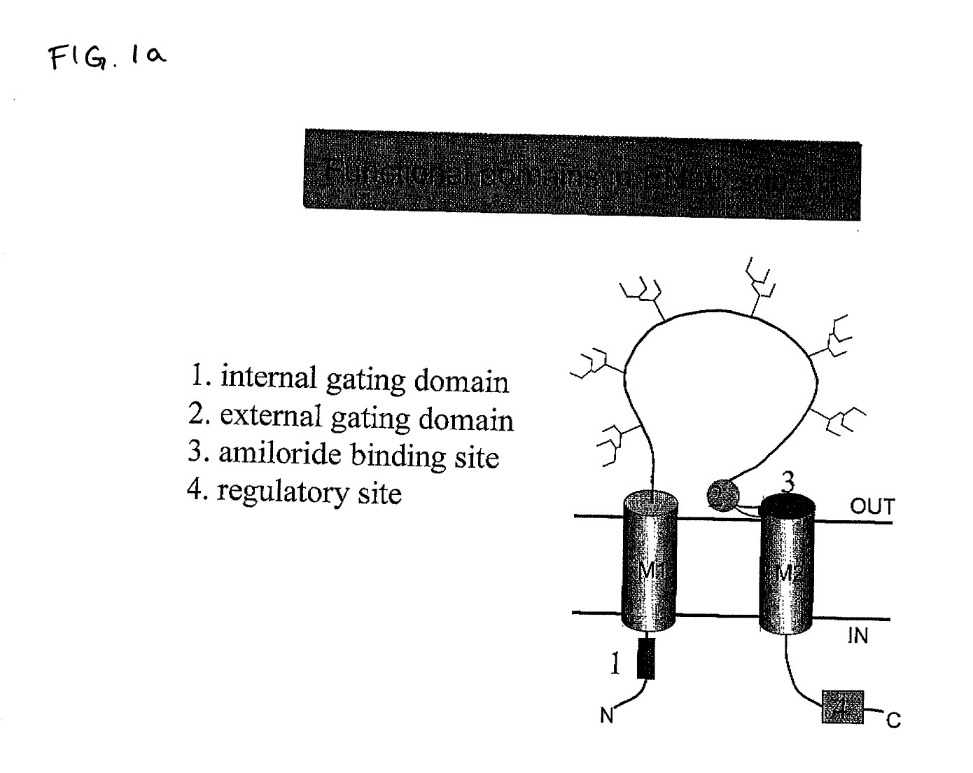

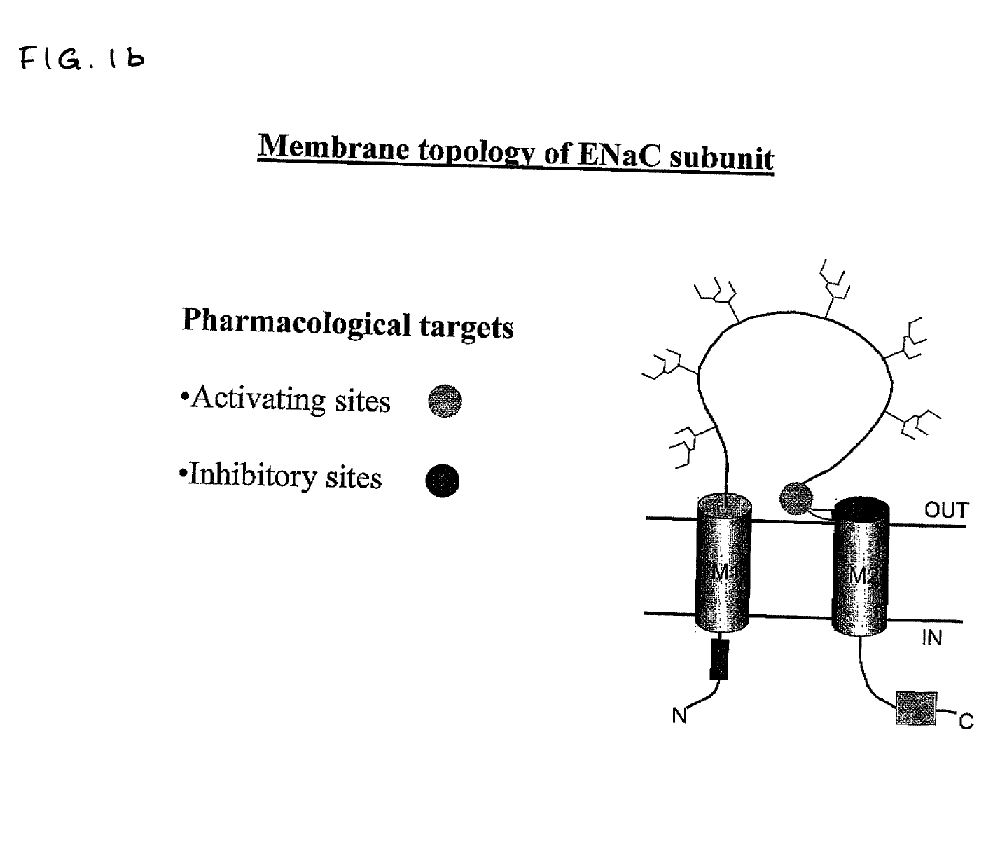

The ENAC is characterized by its high binding affinity to amiloride. Amiloride binds to the extracellular side of the ENAC. (See FIG. 1). The pharmacological profile of amiloride binding makes it possible to distinguish ENAC from other sodium or non-selective cation channels. Moreover, ENAC activity is also inhibited by methanethiosulfonates, which bind to the intracellular side of the channel. The methanethiosulfonates react with the sulfhydryl groups of cysteine residues located on the intracellular side of the ENAC. Methanethiosulfonates include MTSEA (2-aminoethyl Methanethiosulfonate Hydrobromide), MTSET (trimethylamnoiumethyl Methanethiosulfonate Bromide), or MTSES (Sodium (2-sulfonatoethyl) Methanethiosulfonate), as well as larger compounds, including MTS-PTrEA (triethylamoniumpropyl Methanethiosulfonate Bromide). The inhibition of ENAC by the methanethiosulfonates is both complete and irreversible. This indicates covalent binding between the methanethiosulfonates and the sulfhydryl groups of the cysteine residues accessible from the intracellular milieu.

Specific target sequences on each of the ENAC subunits have been identified. These target sequences, which are important for ligand binding and channel inhibition, are found in the N-terminus of the three α, β, and γ ENAC subunits and they are highly conserved between rats and humans. Table 1 depicts each of the target sequences identified on the human ENAC subunits.

TABLE 1
Target Sequences (amino acids conserved between all three subunits are
shown in bold)

| ENAC | | | |
|---|---|---|---|
| Subunit | Location | SEQ ID NO: | Target Sequence |
| α | 69-79 | 1 | HGAIRLVCSQH (SEQ ID NO: 1) |
| β | 36-46 | 2 | HGPKRIICEGP (SEQ ID NO: 2) |
| γ | 39-49 | 3 | HGCRRIVVSRG (SEQ ID NO: 3) |

Identification of these target sequences was accomplished by manipulating the location of a cysteine residue on the β ENAC subunit. In the naturally-occurring protein, the cysteine is located at position 43. It was observed that when sulfhydryl reagents bind to this cysteine (residue C43), they block ENAC activity. When this C43 residue was moved upstream to position R40, the sulfhydryl reagents still bound to the cysteine residue and blocked the ENAC. These data indicate that blocking of the channel is position-independent, i.e., a compound which binds to any one or more residues of SEQ ID NOS: 1, 2, or 3 blocks sodium conductance through an ENAC.

As demonstrated in Table 1, the H, G, and R residues are conserved in each of the three ENAC subunits. In addition, the C residue is conserved between the α and β subunits; the first V residue is conserved between the α and γ subunits; and the S residue is conserved between the α and γ subunits. Thus, an inhibitor of an ENAC binds to a polypeptide containing the amino acid sequence HGXXRXV (SEQ ID NO:4), HGXXRXXC (SEQ ID NO:5), and/or HGXXRXXXS (SEQ ID NO:6).

Ligands binding to any of the amino acids within these target sequences, whether conserved or divergent, block ENAC activity.

An inhibitor of the ENAC binds to an ENAC fragment containing at least one target or at least one consensus target sequence located within an ENAC subunit. Preferably, the ENAC fragment is less than 700 amino acids in length, more preferably less than 500 amino acids in length, more preferably less than 100 amino acids in length, more preferably less than 50 amino acids in length, more preferably less than 25 amino acids in length, or most preferably less than 15 amino acids in length. Additionally, the ENAC fragment is greater than 10 amino acids in length. Alternatively, an ENAC fragment may contain an amino acid sequence having at least 70% homology to one of the target or consensus target sequences. Sequence identity described herein may be measured using the Lasergene software package (DNASTAR, Inc. Madison, Wis.). The MegAlign module used is the Clustal V method (Higgins et al. 1989, CABIOS 5 (2):151-3. The parameters used are gap penalty 10, gap length penalty 10.

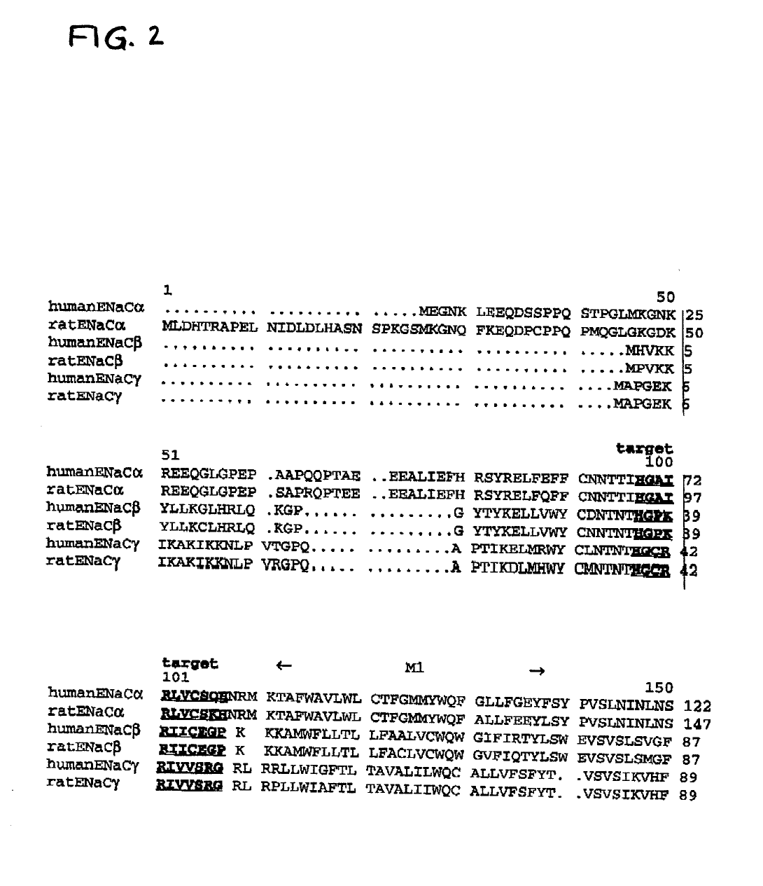

FIG. 2 depicts an alignment of the intracellular N-terminal parts of the ENAC subunits. This figure aligns the intracellular N-terminal portions of human and rat ENAC subunits. The location of the target sequence within each subunit is underlined and bolded. In addition, the first transmembrane segment is indicated as “M1”. The intracellular N-terminal part comprises amino acids 1-83 of the human α subunit, amino acids 1-48 of the human β subunit, and amino acids 1-52 of the human γ subunit.

An ENAC ligand, which binds to a polypeptide containing a target sequence (or a consensus sequence) inhibits ENAC activity. Such inhibitory compounds are useful as diuretics. A compound is a diuretic or has diuretic activity if it inhibits the selective reabsorption of sodium chloride.

As used herein, the term “inhibitory compound” is not intended to encompass amiloride and/or methanethiosulfonates such as MTSEA (2-aminoethyl Methanethiosulfonate Hydrobromide), MTSET (trimethylamnoiunmethyl Methanethiosulfonate Bromide), or MTSES (Sodium (2-sulfonatoethyl) Methanethiosulfonate), as well as larger compounds such as MTS-PTrEA (triethylamonium-propyl Methanethiosulfonate Bromide). Inhibitory compounds include polypeptides, polypeptide fragments, peptide mimetics, small organic molecules, large organic molecules, and/or other drug candidates known to those skilled in the art, which bind or associate with one or more of the ENAC targets described herein.

The invention includes peptide inhibitors in which one or more peptide bonds have been replaced with an alternative type of covalent bond (a “peptide mimetic”), which is not susceptible to cleavage by peptidases. Where proteolytic degradation of the peptides following injection into the subject is a problem, replacement of a particularly sensitive peptide bond with a noncleavable peptide mimetic renders the resulting peptide more stable and thus more useful as a therapeutic. Such mimetics, and methods of incorporating them into peptides, are well known in the art. Similarly, the replacement of an L-amino acid residue is a standard way of rendering the peptide less sensitive to proteolysis. The molecular interactions of a peptide mimetic are similar to that of the naturally-occurring molecule.

An inhibitory compound according to the invention is one that binds directly to one or more of the amino acids of the target or the consensus target sequences. Additionally, an inhibitory compound includes one that binds to an amino acid sequence that is 70% homologous to any of the target sequences or the consensus target sequences. Specifically, an inhibitory compound may bind directly to a cysteine residue in any one or more of SEQ ID NOS: 1,2, or 3. An inhibitory compound may also bind directly to any one or more amino residues contained in any one or more of SEQ ID NOS:1,2, or 3. Likewise, an inhibitory compound may simultaneously or sequentially bind to one or more residues on one or more ENAC subunits.

Preferably, ENAC inhibitory compounds identified according to the methods of the invention bind reversibly to one or more of the target sequences, to one or more of the consensus target sequences, or to one or more sequences that is at least 70% homologous to either the target sequences or the consensus target sequence. A non-limiting example of reversible binding is any noncovalent binding of an inhibitory compound. Any other method of reversible binding known to those skilled in the art is also desirable.

The inhibitors described herein are also useful to other sodium channels, which are homologous to ENAC. For example, brain sodium channel (BNC) is a sodium channel that is homologous to ENAC. It is reversibly inhibited by amiloride and is highly selective for sodium relative to potassium. BNC is a member of the ENAC family. However, differences from other family members include: a lack of discrimination between Na⁺ and Li⁺ current and expression detected only in the central nervous system (CNS). See generally, U.S. Pat. No. 5,892,018, which is incorporated herein by reference. Another member of the ENAC family of sodium channels are acid sensing ion channels (ASIC). ASIC are located in the CNS or in sensory neurons. They are activated by a drop in extracellular pH and likely mediate the pain accompanying tissue acidosis. See Coscoy et al., J. Biol. Chem. 274(15):10129-32 (1999).

Sensory and CNS neurons respond to the application of acidic solutions on their plasma membrane by the opening of cation channels that are sensitive to extracellular protons. See Babinski et al., J. Biol. Chem. 275(37):28519-25 (2000) “Detections of acidosis in the periphery by primary sensory neurons plays a physiological role of protection by informing the central nervous system of noxious conditions of inflammation, hypoxia, or tissue damage.” (Id. at 28519).

ASIC are expressed in brain and in sensory neurons, such as those that innervate the heart. It is likely that ASICs play a role in pain sensation. ASIC 3, which is present in the sensory neurons that innervate the heart, may be involved in ischemic pain.

Binding to sequences, which are at least 70% identical to the ENAC target and conform to consensus ENAC target sequences in BNC and/or ASIC also leads to channel inhibition. The methods disclosed herein are used to identify other compounds that inhibit these related channels.

Screening Assays

ENAC inhibitory compounds can be identified by detecting association or direct binding to a polypeptide containing the amino acid sequence of SEQ ID NO:1, 2, 3, 4, 5, or 6 or by detecting a reduction in current from ENAC using a voltage clamp assay known in the art.

Direct Binding Assays

Candidate inhibitory compounds are screened to identify an inhibitory compound by direct binding to at least one of the target sequences or at least one of the consensus target sequences. This method includes the steps of (a) providing a peptide or peptide fragment containing a target sequence or a consensus target sequence; (b) contacting the target sequence or consensus target sequence with a candidate inhibitory compound; and (c) determining whether the candidate compound binds to the target sequence or the consensus target sequence. Alternatively, the candidate compound may be contacted with the target or consensus target sequence. In another variation of the assay, the target or consensus target sequences and the candidate compound may be incubated together simultaneously, followed by a measurement of binding.

The assays described herein are utilized as a high throughput assay to screen peptide libraries, natural product libraries, pharmaceutical compound files, or any panel of compounds to identify new classes of compounds, which bind to an ENAC target site described above. A standard direct binding assay is used to identify candidate compounds which associate with or directly bind to a polypeptide containing the amino acid sequence of SEQ ID NO:1, 2, or 3 or a polypeptide which conforms to the consensus sequence SEQ ID NO:4, 5, or 6.

To perform a binding assay, the peptide is labeled with a detectable marker such as a radioligand (e.g., ³H, ³⁵S, ¹²⁵I). The candidate compound or panel of compounds (e.g., a peptide library, recombinant DNA expression library, panel of organic compounds etc.) are immobilized. For example, the candidate compound may be spotted onto a filter, e.g., a nitrocellulose or nylon fiter, or immobilized on a test tube. The labeled peptide (containing an amino acid sequence of SEQ ID NO:1, 2, 3, 4, 5, or 6) is added to the candidate compound(s) in a buffer system suitable for binding, e.g., phosphate buffered saline. The labeled peptide and compound are then allowed to bind. Nonspecific binding is determined in the presence of unlabelled peptide. Samples are incubated under standard assay conditions, e.g., 0-4 degrees Centrigrade for 1 hour. The filters are washed and peptide binding is evaluated by measuring presence of the detectable marker on the immobilized candidate compound. For example, binding of a radiolabeled target peptide sequence is detected by measuring radioactivity using a scintillation counter. Detection of radioactivity (or any another detectable marker) at a site of a certain candidate compound indicates that the candidate compound associates with or directly binds to the target peptide and that the compound inhibits ENAC activity.

Other direct binding assays known in the art may also be used. For example, a peptide or peptide fragment containing at least one of the target or consensus target sequences is immobilized prior to contact with the candidate inhibitory compound. The peptides can be immobilized using methods known in the art, such as adsorption onto a plastic 96-well microtiter plate, an array, a computer chip, or a column, or via specific binding of a GST-fusion protein to a polymeric bead containing glutathione. For example, GST fused to a peptide or a peptide fragment containing at least one of the target or consensus target sequences can be bound to glutathione-Sepharose beads. The immobilized peptide is then contacted with a labeled candidate inhibitory compound. Alternatively, the immobilized peptide may be labeled and then contacted with a candidate inhibitory compound.

Any unbound candidate inhibitory compound is removed by any method commonly employed in the art. Next, the eluted material is analyzed to determine whether any of the candidate inhibitory compound bound to the immobilized peptide or peptide fragment.

Another screening method that may be employed is a random peptide library screening method. As used herein, the term “random peptide library” refers to a collection of potential candidate inhibitory peptides that have been immobilized. In one embodiment of this method, the potential candidate peptides are immobilized by plating on replicate nitrocellulose filters. Subsequently, labeled peptides or peptide fragments containing at least one of the target or consensus target sequences are contacted with and allowed to bind to the filters. Alternatively, the immobilized potential candidate inhibitory peptides may be labeled. After allowing for sufficient time for binding, any unbound peptide is washed away, and the filters are analyzed for target and/or consensus target sequence binding. Binding indicates that the potential candidate inhibitory peptide directly binds to the target or consensus target sequence. Thus, the bound peptide is a candidate inhibitory compound.

Cellular Assays

Cellular assays are used for primary or secondary screens of candidate compounds for their ability to block ENAC activity. For example, once a candidate inhibitory compound has been shown to bind one or more of the target or consensus target sequences, a cellular assay is carried out to determine whether the compound inhibits conductance through ENAC. Cellular assays that may be employed include, for example, the cut-open oocyte technique, sodium transport measurements in cultured cell lines, and circular dichroism. However, any other cellular assay known in the art may also be employed.

Cut-Open Oocyte Technique

One example of a cellular assay that can be used to further screen candidate inhibitory compounds is the cut-open oocyte technique which is known in the art. This technique is set forth in Abriel et al., “Feedback inhibition of rat amiloride-sensitive epithelial sodium channels expressed in Xenopus laevis oocytes,” The Journal of Physiology 561(l):31-43 (1999), which is incorporated herein by reference. Xenopus oocytes are internally perfused using an intracellular perfusion pipette, thereby allowing application of candidate ENAC inhibitors from the intracellular side of the channel. The extracellular bathing medium may be rapidly exchanged thereby allowing application of amiloride, or another specific ENAC blocker acting from the extracellular side of the ENAC.

Sodium current passing through the ENAC in the oocyte membrane is recorded using standard methods of measuring current and voltage. In an assay to determine whether a candidate inhibitory compound binds to the ENAC, the amount of current passing through an ENAC that is exposed to a candidate inhibitory compound is measured. Likewise, the amount of current passing through a channel not exposed to the candidate compound is also measured. A comparison of the amount of current passing through each of these channels is used to determine whether the candidate compound inhibits ENAC activity. A decrease in the amount of current passing through the ENAC, as compared to the control channel, indicates that ENAC activity is inhibited by the candidate compound.

Sodium Transport Measurements in Cultured Cell Lines

Kidney cell lines expressing ENAC are grown on permeable filters. Transepithelial sodium transport will be measured by short circuit measurements and transepithelial electrical resistances by any method known to those of ordinary skill in the art, such as that of Bens et al., J. Am. Soc. Nephrol. 10:923-34 (1999). ENAC blockers applied either on the apical or basolateral membrane that will change sodium transport across the cell epithelium will also affect electrical short circuit current and transepithelial electrical resistance. Alternatively, changes in cellular sodium transport induced by ENAC blockers will depolarize the apical cell membrane. This membrane depolarization can be detected by any means known to those skilled in the art, including, but not limited to the methods described in J. Physiol. 517:781 (1999) and Br. J. Pharmacol. 129:1323 (2000).

Using this technique, candidate compounds are screened to determine whether a candidate inhibitory compound binds to the ENAC. For example, transepithelial sodium transport through an ENAC that is exposed to a candidate inhibitory compound is measured. Likewise, the amount of transepithelial sodium transport through a channel not exposed to the candidate compound is also measured. A comparison of the amount of transepithelial sodium transport through each of these channels is used to determine whether the candidate compound inhibits ENAC activity. A decrease in the amount of transepithelial sodium transport through the ENAC, as compared to the control channel, indicates that ENAC activity is inhibited by the candidate compound.

Circular Dichroism

Circular dichroism (CD) is a spectroscopic parameter capable of giving information about an individual optically-active absorption band at a wavelength specific for that chromophore (e.g., for example, an electron transition of a helix at 222 nm). The interaction of light with an asymmetric molecule results in a preferential interaction of one circularly polarized component which, in an absorption region, will be seen as a differential absorption (i.e., a dichroism). See Urry, D. W., American Medical Association Press, Chicago, Ill., pp 33-120 (1969).

CD is an absorptive phenomenon that results when a chromophore interacts with plane polarized light at a specific wavelength. The absorption band can be either negative or positive depending on the differential absorption of the right and left circularly polarized components for that chromophore. Unlike optical rotatory dispersion (ORD), which measures the contributions of background and the chromophore of interest many millimicrons from the region of actual light interaction, CD offers the advantage of measuring optical events at the wavelength at which the event takes place, and, thus, is specific to the electronic transition of the chromophore. See Beychok, S., Science, 154:1288-1299 (1966). An example of a circular dichroism screening assay is described in detail in U.S. Pat. No. 5,780,242, which is incorporated herein by reference.

Application of circular dichroism to solutions of macromolecules has resulted in the ability to identify conformation states (Jirgensons, B., Springer-Verlay, Berlin, Germany, pp. 20-39, 1969, and Gratzer, W. B. et al., Nature, (see Beychok, supra) 222:426-431 (1969). The technique can distinguish random coil, alpha helix, and beta chain conformation states of macromolecules.

A change in the circular dichroism of ENAC in the presence of a candidate compound compared to that in the absence of the compound indicates that the compound alters the conformation of ENAC and inhibits the channel.

Other methods of measuring sodium channel activity are know in the art, e.g., the method described in detail in U.S. Pat. No. 5,437,982, which is herein incorporated by reference.

Methods of Treatment

Compounds, which bind to SEQ ID NO:1, 2, 3, 4, 5, or 6 are used to treat a variety of conditions characterized by irregular ENAC activity. Examples of such conditions include hypertension, renal insufficiency, any electrolyte imbalance, cystic fibrosis, Liddle's syndrome, as well as any other diseases and/or syndromes known by those skilled in the art to be caused by or result from irregular or inappropriate ENAC activity.

Inhibitory compounds, which reversibly bind to an ENAC target sequence or a consensus target sequence, possess advantages over current methods of treating diseases characterized by irregular ENAC activity. These advantages include, but are not limited to, lower toxicity and the ability to regulate the extent of ENAC inhibition.

The inhibitory compounds of the invention, and derivatives, fragments, analogs and homologs thereof, can be incorporated into pharmaceutical compositions suitable for administration. Such compositions typically comprise the inhibitory compound and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an inhibitory compound) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

Inhibitory compounds are optionally prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specifications for the dosage unit forms of the invention are dictated by and directly dependent on the unique characteristics of the active compound and the particular therapeutic effect to be achieved.

The pharmaceutical compositions can be included in a container, pack, or dispenser together with instructions for administration.

ENAC inhibitory compounds are useful for reducing hypertension in a patient suffering therefrom. The compounds are also useful to treat renal insufficiency and other conditions related to an electrolyte imbalance. An inhibitory compound is administered in a manner similar to those of conventional hypertension drugs such as diuretics, antiadrenergic agents, vasodilators, angiotensin-converting enzyme inhibitors, and calcium channel agonists. Any methods of administering anti-hypertensive agents that are known to those of ordinary skill in this art can be applied can be used with the treatment methods of this invention. ENAC inhibitory compounds may be administered to a mammal in need of treatment. Suitable mammals include, but are not limited to, humans, cows, pigs, horses, and dogs.

A number of agents are known for the treatment of hypertension. Certain of these, for example, reserpine, are effective in lowering the blood pressure in some patients, but in other patients, they give rise to undesirable and well-known side effects such as central nervous system depression.

The inhibitory compounds of the present invention exhibit useful and potent anti-hypertensive activity. Further, the inhibitory compounds of the invention are active as anti-hypertensive agents, while avoiding or mitigating the deleterious side effects, such as irreversibility and/or difficulty in regulating the response to the inhibitory compound, associated with known anti-hypertensive agents.

An effective dosage (the ED₅₀) by oral administration for a compound of the present invention is within the range of from 0.05 to 20 mg/kg of mammalian weight. For example, daily human dose is about 0.1 to 500 mg. The required daily dosage may be administered in single or divided doses. The exact dose to be administered will, of course, be dependent upon where the compound in question lies within the above quoted dosage ranges and upon the age and weight of the subject mammal.

The inhibitory compounds may be administered orally. In any event, a suitable pharmaceutical carrier is employed, with the carrier selected according to the physical properties of the compound in the pharmaceutical composition. The carrier should not react chemically with the inhibitory compound to be administered. The preparations containing the active ingredients may typically be in the form of tablets, capsules, syrups, elixirs or suspensions.

In treating certain patients with the inhibitory compounds of this invention, it may be desirable to include other pharmaceutically active ingredients in the same dosage unit. For example, in treating patients in whom salt and water retention is a problem, effective amounts of conventional diuretics can be incorporated, such as the thiazide diuretics, e.g., hydrochlorothiazide or trichloromethiazide.

A typical antihypertensive composition within the scope of the present invention contains from about 0.2 to about 500 mg of the derivative of the present invention or a physiologically acceptable salt thereof or a mixture thereof blended with a physiologically acceptable vehicle, carrier, extender, binder, antiseptic, stabilizer, flavoring agent or the like in a unit amount as required for a conventional pharmaceutical preparation. The amount of the active ingredient in the pharmaceutical composition or preparation should be such that an appropriate dosage falling within the indicated range can be obtained by the administration of the said composition or preparation.

Examples of pharmaceutical excipients which are combined with the present antihypertensive agent for the preparation of tablets, capsules and the like include binders such as tragacanth, gum arabic, corn starch or gelatin; a vehicle such as fine crystalline cellulose; an extender such as corn starch, pre-gelatinated starch, alginic acid, or the like; a sweetener such a sucrose, lactose, or saccharin; a flavoring agent such as peppermint, an oil from Gaulthenia adenothrix Maxim or cherry. When the unit preparation is in the form of a capsule, the composition may further contain a liquid carrier such as a fat or oil, in addition to the above-mentioned additive materials. Other ingredients may be employed which form coated pills or which vary the physical form of the unit preparation by a different method. For example, tablets can be coated with shellac, sugar or a combination thereof. A syrup or elixir can contain the active compound together with sucrose as a sweetener, methyl- or proply-paraben as an antiseptic, a dye and cherry or orange aroma as a flavoring agent.

Anti-ENAC Polypeptide Antibodies

The invention also encompasses antibodies and antibody fragments, such as F_{ab} or (F_{ab})2, that bind immunospecifically to any of the ENAC polypeptides of said invention.

An isolated ENAC polypeptide, or a portion or fragment thereof, can be used as an immunogen to generate antibodies that bind to ENAC polypeptides using standard techniques for polyclonal and monoclonal antibody preparation. The full-length ENAC polypeptides can be used or, alternatively, the invention provides antigenic peptide fragments of ENAC polypeptides for use as immunogens. The antigenic ENAC peptides comprises at least 4 amino acid residues of the amino acid sequence shown SEQ ID NOS:1, 2, 3, 4, 5, or 6 and encompasses an epitope of ENAC polypeptide such that an antibody raised against the peptide forms a specific immune complex with ENAC. Preferably, the antigenic peptide comprises at least 6, 8, or 10 amino acid residues. Longer antigenic peptides are sometimes preferable over shorter antigenic peptides, depending on use and according to methods well known to someone skilled in the art.

As disclosed herein, ENAC polypeptides sequences of SEQ ID NOS: 1, 2, 3, 4, 5, or 6, or derivatives, fragments, analogs or homologs thereof, may be utilized as immunogens in the generation of antibodies that immunospecifically-bind these protein components. The term “antibody” as used herein refers to immunoglobulin molecules and immunologically-active portions of immunoglobulin molecules, i.e., molecules that contain an antigen binding site that specifically-binds (immunoreacts with) an antigen, such as an ENAC polypeptide. Such antibodies include, but are not limited to, polyclonal, monoclonal, chimeric, single chain, F_{ab} and F_{(ab′)2} fragments, and an F_{ab} expression library. In a specific embodiment, antibodies to human ENAC polypeptides are disclosed. Various procedures known within the art may be used for the production of polyclonal or monoclonal antibodies to an ENAC polypeptide sequence of SEQ ID NOS:1, 2, 3, 4, 5, or 6, or a derivative, fragment, analog or homolog thereof.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly-expressed ENAC polypeptide or a chemically-synthesized ENAC polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against an ENAC polypeptide can be isolated from the mammal (e.g., from the blood) and further purified by well-known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of an ENAC polypeptide. A monoclonal antibody composition thus typically displays a single binding affinity for a particular ENAC polypeptide with which it immunoreacts. For preparation of monoclonal antibodies directed towards a particular ENAC polypeptide, or derivatives, fragments, analogs or homologs thereof, any technique that provides for the production of antibody molecules by continuous cell line culture may be utilized. Such techniques include, but are not limited to, the hybridoma technique (see, e.g., Kohler & Milstein, 1975. Nature 256: 495-497); the trioma technique; the human B-cell hybridoma technique (see, e.g., Kozbor, et al., 1983. Immunol. Today 4: 72) and the EBV hybridoma technique to produce human monoclonal antibodies (see, e.g., Cole, et al., 1985. In: MONOCLONAL ANTIBODIES AND CANCER THERAPY, Alan R. Liss, Inc., pp. 77-96). Human monoclonal antibodies may be utilized in the practice of the invention and may be produced by using human hybridomas (see, e.g., Cote, et al., 1983. Proc Natl Acad Sci USA 80: 2026-2030) or by transforming human B-cells with Epstein Barr Virus in vitro (see, e.g., Cole, et al., 1985. In: MONOCLONAL ANTIBODIES AND CANCER THERAPY, Alan R. Liss, Inc., pp. 77-96). Each of the above citations is incorporated herein by reference in their entirety.

According to the invention, techniques can be adapted for the production of single-chain antibodies specific to an ENAC polypeptide (see, e.g., U.S. Pat. No. 4,946,778). In addition, methods can be adapted for the construction of F_{ab} expression libraries (see, e.g., Huse, et al., 1989. Science 246: 1275-1281) to allow rapid and effective identification of monoclonal F_{ab} fragments with the desired specificity for an ENAC polypeptide or derivatives, fragments, analogs or homologs thereof. Non-human antibodies can be “humanized” by techniques well known in the art. See, e.g., U.S. Pat. No. 5,225,539. Antibody fragments that contain the idiotypes to an ENAC polypeptide may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent; and (iv) Fᵥ fragments.

Additionally, recombinant anti-ENAC polypeptide antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No. 184,187; European Patent Application No. 171,496; European Patent Application No. 173,494; PCT International Publication No. WO 86/01533; U.S. Pat. Nos. 4,816,567; 5,225,539; European Patent Application No. 125,023; Better, et al., 1988. Science 240: 1041-1043; Liu, et al., 1987. Proc. Natl. Acad. Sci. USA 84: 3439-3443; Liu, et al., 1987. J. Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314 :446-449; Shaw, et al., 1988. J. Natl. Cancer Inst. 80: 1553-1559); Morrison (1985) Science 229:1202-1207; Oi, et al. (1986) BioTechniques 4:214; Jones, et al., 1986. Nature 321: 552-525; Verhoeyan, et al., 1988. Science 239: 1534; and Beidler, et al., 1988. J. Immunol. 141: 4053-4060. Each of the above citations are incorporated herein by reference in their entirety.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an ENAC polypeptide is facilitated by generation of hybridomas that bind to the fragment of an ENAC polypeptide possessing such a domain. Thus, antibodies that are specific for a desired domain within an ENAC polypeptide, or derivatives, fragments, analogs or homologs thereof, are also provided herein.

Anti-ENAC polypeptide antibodies may be used in methods known within the art relating to the localization and/or quantitation of an ENAC polypeptide (e.g., for use in measuring levels of the ENAC polypeptide within appropriate physiological samples, for use in diagnostic methods, for use in imaging the protein, and the like). In a given embodiment, antibodies for ENAC polypeptides, or derivatives, fragments, analogs or homologs thereof, that contain the antibody derived binding domain, are utilized as pharmacologically-active compounds. An anti-ENAC polypeptide antibody (e.g., monoclonal antibody) can be used to isolate an ENAC polypeptide by standard techniques, such as affinity chromatography or immunoprecipitation. An anti-ENAC polypeptide antibody can facilitate the purification of natural ENAC polypeptide from cells and of recombinantly-produced ENAC polypeptide expressed in host cells. Moreover, an anti-ENAC polypeptide antibody can be used to detect ENAC polypeptide in order to evaluate the abundance and pattern of expression of the ENAC polypeptide. Anti-ENAC polypeptide antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

### EXAMPLES

The invention will be further described in the following examples, which do not limit the scope of the invention described in the claims.

### Example 1

### Cellular Assay: Cut-Open Oocyte Technique

ENAC inhibitory activity is measured using the “cut-open oocyte technique”. This technique is well known in the art and set forth in Abriel et al., “Feedback inhibition of rat amiloride-sensitive epithelial sodium channels expressed in Xenopus laevis oocytes,” The Journal of Physiology 561(1):31-43 (1999), which is incorporated herein by reference.

ENAC expression in Xenopus oocytes

The rat ENAC was expressed in Xenopus oocytes by coinjection of mRNAS coding for the three α, β and γ ENAC subunits. Messenger RMA were obtained from in vitro transcription of α, β, and γ ENAC cDNAs. See Canessa et al., Nature 361: 467-70 (1993); Canessa et al., Nature 367: 463-67 (1994). The human ENAC (hENAC) genes have been isolated and share 80 to 85% homology with the rat ENAC genes. See McDonald et al., Am. J. Physiol. Cell Physiol. 268:C1157-63 (1995); Voilley et al., Comparative Biochemistry & Physiology 1997:193-200 (1994); and Voilley et al., Proc. Natl. Acad. Sci. USA 91:247-51 (1994).

ENAC activity was determined by electrophysiological measurements of the Na current through ENAC channels. This ENAC mediated Na current was recognized by its sensitivity to a specific blocker of ENAC amiloride, see Schild et al., J. Gen. Physiol. 109:15-26 (1997).

Xenopus oocytes were internally perfused using an intracellular perfusion pipet allowing application of channel inhibitors from the intracellular side of the channel. The extracellular bathing medium could be rapidly exchanged allowing application of amiloride, a specific ENAC blocker acting from the extracellular side. Na current through ENAC in the oocyte membrane were recorded using the cut-open technique originally developed by Taglialatela et al., Biophys. J. 61:78-82 (1992). See also Abriel et al., J. Physiol. (Lond.) 516:31-43 (1999).

Stage V-VI oocytes are surgically removed from the ovarian tissue of female Xenopus laevis, which had been anaesthetized by immersion in MS-222 (2 g I-1; Sandoz, Basel, Switzerland). Following surgery, the frogs are allowed to recover in isolation in a shallow tank, and, after full recovery had been verified a few hours later, they are returned to the rearing tank. About two months later, the frogs are operated on a second time for the removal of the ovarian lobe on the other side. The Xenopus are then killed by decapitation under anesthesia. All procedures are performed in accordance with local institutional animal welfare guidelines. The oocytes are defolliculated as described previously (Puoti et al. Am. J. Physiology 38:C188-197 (1995)) and are pressure-injected at the border between the vegetal and animal poles with 50 nl of a solution containing equal amounts of the cRNAs of the rENAC subunits (total quantity, 10 ng per oocyte). The site of injection is chosen so as not to injure the vegetal pole. After injection, the oocytes are kept in modified Barth's solution (MBS) containing 1 mM Na+ to prevent an increase in [NA+] and thereby allow observation of sodium-dependent downregulation. Electrophysiological measurements are performed at room temperature (20-25° C.), 14-40 h after cRNA injection.

Electrophysiological Measurements

The cut-open oocyte technique, which was originally developed by Taglialatela et al., Biophysical Journal 61:78-82 (1992), is used for the electrophysiological measurements. A Xenopus oocyte is mounted between two compartments with the studied vegetal pole upwards, since preliminary experiments show a larger current at this pole. The superior pole of the oocyte is in contact with the upper bath through a hole of 500 um in diameter. The middle (guard) bath serves to provide electrical isolation between the upper (extracellular) and lower (intracellular) compartments through independent voltage clamping of the middle bath at the same electrical potenial as the upper bath. The upper (extracellular) compartment is superfused by gravity (flow rate, 6 ml min⁻¹) with an extracellular sodium-containing solution.

The lower pole of the oocyte is impaled with a glass microelectrode which is simultaneously used as an intracellular perfusion pipette and a voltage-recording electrode. This modification of the original set-up was first described by Costa et al., Biophysical Journal 67:395-401 (1994). The resistance of the electrode, when filled with the intracellular solutions described below, was about 0.2-0.7 M. For the purpose of intracellular perfusion, the pipette is advanced into the oocyte until it is just visible from above through the membrane and yolk.

The flow rate for perfusion is chosen so that a “washing-out” of the yolk platelets could be observed. With time, the membrane becomes translucent. Only oocytes in which this is observed are considered to be intracellularly well perfused and only these are used for further experiment and analysis. To obtain this effect, the flow rate needs to between 1 and 6 ul min⁻¹. Higher rates for perfusion almost always cause a rapid and marked loss of membrane resistance or create visible holes in the membrane. The solution is perfused by means of a precision syringe pump (Infors AG, Basel, Switzerland). In order to minimize the dead space when the perfusion solution is changed, two thin capillaries are introduced by which test solutions are introduced into the perfusion pipette close to the tip. The remaining dead space is about 2-5 μl. The voltage clamp is performed using a Dagan cut-open oocyte voltage-clamp apparatus (Dagan Corporation, Minneapolis, Miss., USA; Model CA-2 High Performance Oocyte Clamp).

Determination of Binding

Sodium current passing through the ENAC in the oocyte membrane is recorded using this technique. To determine whether a candidate inhibitory compound binds to the ENAC, the amount of current passing through an ENAC that is exposed to a candidate inhibitory compound is measured and compared to the amount of sodium current passing through a “control” ENAC that is not exposed to any candidate inhibitory compound(s). A decrease in the amount of current passing through the ENAC, as compared to the control channel, indicates that ENAC activity is inhibited by the candidate compound.

### OTHER EMBODIMENTS

Although particular embodiments have been disclosed herein in detail, this has been done by way of example for purposes of illustration only, and is not intended to be limiting with respect to the scope of the appended claims that follow. In particular, it is contemplated by the inventor that various substitutions, alterations, and modifications may be made to the invention without departing from the spirit and scope of the invention as defined by the claims. For example, the selection of the inhibitory compound that is to be utilized in the practice of the present invention is believed to be a matter of routine for a person of ordinary skill in the art with knowledge of the embodiments described herein.

What is claimed is:

1. A composition comprising a sodium channel inhibitor, wherein said inhibitor binds to a polypeptide comprising the amino acid sequence of HGXXRXV (SEQ ID NO:4), HGXXRXXC (SEQ ID NO:5), or HGXXRXXXS (SEQ ID NO:6).

2. The composition of claim 1 comprising the amino acid sequence of HGXXRXV (SEQ ID NO:4).

3. The composition of claim 1 comprising the amino acid sequence of HGXXRXXC (SEQ ID NO:5).

4. The composition of claim 1 comprising the amino acid sequence of HGXXRXXXS (SEQ ID NO:6).

5 The composition according to claim 1, wherein said inhibitor is not amiloride or a methanethiosulfonate.

6. The composition of claim 1, wherein said polypeptide comprises the amino acid sequence of HGAIRLVCSQH (SEQ ID NO:1), HGPKRIICEGP (SEQ ID NO:2), or HGCRRIVVSRG (SEQ ID NO:3).

7. The composition of claim 4 comprising the amino acid sequence of HGAIRLVCSQH (SEQ ID NO:1).

8. The composition of claim 4 comprising the amino acid sequence of HGPKRIICEGP (SEQ ID NO:2).

9. The composition of claim 4 comprising the amino acid sequence of HGCRRIVVSRG (SEQ ID NO:3).

10. The composition of claim 1, wherein said polypeptide comprises an amino acid sequence that is 70% identical to HGAIRLVCSQH (SEQ ID NO:1), HGPKRIICEGP (SEQ ID NO:2), or HGCRRIVVSRG (SEQ ID NO:3), and wherein a non-identical amino acid is a conservative substitution.

11. A pharmaceutical composition comprising the composition of claim 1 and a pharmaceutically acceptable carrier.

12. A kit comprising the composition of claim 1.

13. A method of identifying a sodium channel inhibitor, comprising contacting an ENAC polypeptide comprising the amino acid sequence of SEQ ID NO:1 with a candidate compound and determining whether said candidate compound binds to said ENAC polypeptide, wherein binding of said compound indicates that said compound inhibits a sodium channel.

14. A method of identifying a sodium channel inhibitor, comprising contacting an ENAC polypeptide comprising the amino acid sequence of SEQ ID NO:2 with a candidate compound and determining whether said candidate compound binds to said ENAC polypeptide, wherein binding of said compound indicates that said compound inhibits a sodium channel.

15. A method of identifying a sodium channel inhibitor, comprising contacting an ENAC polypeptide comprising the amino acid sequence of SEQ ID NO:3 with a candidate compound and determining whether said candidate compound binds to said ENAC polypeptide, wherein binding of said compound indicates that said compound inhibits a sodium channel.

16. A method of reducing hypertension in a mammal, comprising administering to said mammal the composition of claim 1.

17. The method of claim 16, wherein the mammal is a human.

18. The method of claim 16, wherein said mammal is suffering from or at risk for developing salt-sensitive hypertension.

19. The method of claim 16, further comprising administering a conventional diuretic.

20. A method of reducing hypertension in a mammal comprising administering the pharmaceutical composition of claim 11 to the mammal.

21. A method of treating a disorder associated with an electrolyte imbalance in a mammal, comprising administering to said mammal the composition of claim 1.

22. The method of claim 21, wherein the mammal is a human.

23. The method of claim 21 further comprising administering a conventional diuretic.

24. The method of claim 21, wherein the disorder associated with an electrolyte imbalance is selected from the group consisting of hypertension or renal insufficiency.

25. A method of treating a disorder associated with abnormal ENAC activity in a mammal, comprising administering to said mammal the composition of claim 1.

26. The method of claim 25, wherein the mammal is a human.

27. The method of claim 25, wherein the disorder is selected from the group consisting of hypertension, renal insufficiency, an electrolyte imbalance, cystic fibrosis, and Liddle's syndrome.
